(19) United States
(12) Patent Application Publication
(10) Pub. No.: US 2015/0182535 A1
(43) Pub. Date: Jul. 2, 2015

(54) Antimicrobial Composition for the Treatment of Methicillin-resistant Staphylococcus aureus

(71) Applicant: Peter S. Parsonson, Dallas, TX (US)

(72) Inventor: Peter S. Parsonson, Dallas, TX (US)

(21) Appl. No.: 14/329,969

(22) Filed: Jul. 13, 2014

Related U.S. Application Data
(60) Provisional application No. 61/922,889, filed on Jan. 2, 2014.

(51) Int. Cl. A61K 31/541; A61K 31/70; A61K 31/5383; A61K 38/14; A61K 31/351; A61K 31/43; A61K 31/4174; A61K 31/7036; A61K 31/496

(52) U.S. Cl. CPC A61K 31/541; A61K 31/7036; A61K 31/70; A61K 31/5383; A61K 31/496; A61K 31/351; A61K 31/43; A61K 31/4174; A61K 38/14

(57) ABSTRACT

Antibiotic resistance in bacteria may be an inherent trait or may be acquired by mutation. Bacteria that are resistant to antibiotics have become a serious public health threat. For example, approximately 1% of the population in the world has methicillin-resistant Staphylococcus aureus (MRSA), a bacterial strain that is resistant to commonly used antibiotics. Most MRSA infections occur in hospitals and healthcare facilities, such as nursing homes and dialysis centers. It is known as heath-associated MRSA (HA-MRSA). Elderly people or people with weakened immune systems are at a high risk of HA-MRSA infection. Recently, among otherwise healthy people in a wider community, another type of MRSA, community-associated MRSA (CA-MRSA), has been found. CA-MRSA is responsible for serious skin and soft tissue infections and for a serious form of pneumonia. Infection caused by antibiotic-resistant bacteria is often incurable by existing antibiotics. Thus, there is a need to develop new antibiotic drugs.

This invention relates to one certain extract of the Artemisia annua plant, both in its crude and refined form composed substantially of Artemisone, a compound that is chemically classified as a sesquiterpene with an endo-peroxide group [FIG. 1]. This compound is suitable for compositions comprising topical application as well as oral applications for the treatment of bacterial infections caused by Methicillin-resistant Staphylococcus aureus.

### BRIEF SUMMARY OF THE INVENTION

This invention relates to a method of treating infection caused by methicillin-non-susceptible bacteria, vancomycin non-susceptible bacteria, penicillin-non-susceptible bacteria, clarithromycin-non-susceptible bacteria, or metronidazole-non-susceptible bacteria. The method includes topical administration to a subject of an effective amount of artemisone in conjuction with standard antibiotics such as gentamycin, clindamycin, finafloxacin, ofloxacin, ciprofloxacin, tobramycin, amikacin, mupirocin, penicillin, clarithromycin, metronidazole, cephalosporin, nafcillin, or vancomycin.

### BRIEF DESCRIPTION OF THE DRAWINGS

FIG. 1. FIG. 1 shows the chemical structure of Artemisone and its endoperoxide bridge.

### DETAILED DESCRIPTION AND BEST MODE OF IMPLEMENTATION

This application claims priority to U.S. Provisional Application No. 61/922,889, filed Jan. 2, 2014 which is incorporated herein by reference in their entireties.

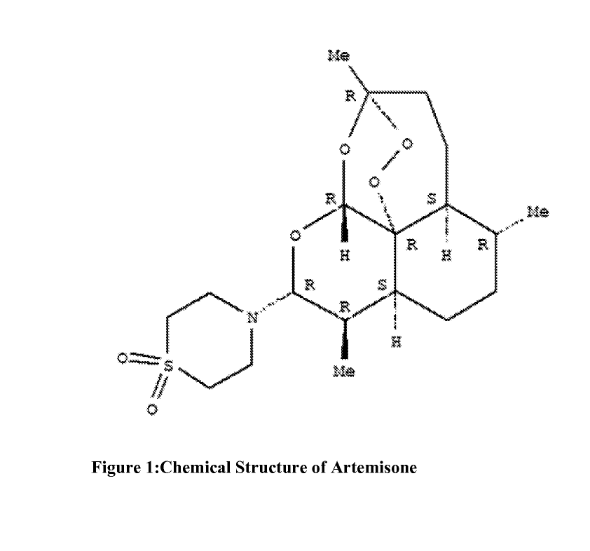

This invention relates to certain extracts of the Artemisia annua plant, both in their crude and refined forms, and certain refined forms of Artemisia annua plant extracts composed substantially of Artemisone FIG. 1, which is chemically classified as asesquiterpene containing an endo-peroxide bridge. The compound is suitable for topical application and for oral administration with other common antibiotics for the treatment Methicillin-resistant Staphylococcus aureus.

The present invention discloses a method of topical parenteral, or oral administration of Artemisone as a refined form from the Artemisia annua plant composed substantially of Artemisone. This method can include a base, a carrier, a solvent or a delivery system.

Artemisia annua extracts have been used in ancient Chinese medicine (qinqhao) for a number of treatments. Medicinal use of the Chinese herb qinqhao appears in several standard Chinese Materia Medica texts as a treatment for febrile illnesses. The herb was specifically recommended for fevers in the Zhou Hou Bei fi Fang (The Handbook of Prescriptions for Emergencies) written by Ge Heng and published in 341 AD. The most detailed description appears in the “Compendium of Materia Medica—Ben Cao Gang Mu, compiled in 1596, and is still printed in China today. The antimalarial activity of qinqhao was rediscovered in China in 1972, and the antimalarial active principal of qinqhao was named “qinghaosu”. The western name for the compound is artemisinin. Recently, however, this extract and its sesquiterpene derivatives have been used for the treatment of malaria. Distribution of artemisinin in Artemisia annua has been reviewed [Ferreira et al., Progress in New Crops, J. Janick (ed.), ASHS Press 579 (1996)].

Moreover, it has been disclosed that Artemisone FIG. 1 is a highly potent and active agent in delivering the antimalarial benefits of Artemisia annua extracts. Only orally administered compositions seem to provide antimalarial benefits.

Artemisinin (Qinghaosu) and its analogs including Artemisone are the treatments of choice for cerebral or chloroquine resistant malaria or for patients with chloroquine allergy. Artemisone is a sesquiterpene lactone with a peroxide bridge, and is characterized by very low toxicity. Artemisone stimulates cell-mediated immunity, and yet decreases abnormally elevated levels of polyamine regulatory proteins. It also markedly inhibits nucleic acid and protein syntheses. Further, it affects cellular membrane functions and decreases hepatic cytochrome oxidase enzyme system activity. Still further, it is virustatic against influenza and cidal against three groups of pathogenic parasites.

The very low toxicity of this compound to humans is a major benefit. For example, artesunate is twice as safe as artemether and only one-fiftieth as toxic as chloroquinine, the most common antimalarial. The first manifestation of toxicity of these compounds is generally a decreased reticulocyte count. Other manifestations include transient fever, decreased appetite and elevated blood transaminase levels, the latter an indication of hepatotoxicity.

In the present invention it is thus both surprising and unexpected that Artemisone can be utilized in topical and orally administered formulations as a treatment for Methicillin-resistant Staphylococcus aureus.

Moreover, the inclusion of iron, especially in a complexed form, further increases the efficacy of the compositions of the present invention. This is contrary to traditionally practiced antibiotic treatments', such as tetracycline and macrolide

What is claimed is:

1. The use of Artemisone as a treatment for bacterial infections caused by Methicillin-resistant Staphylococcus aureus. The claimed treatment is to be administered in conjunction with other antibiotics including the following: gentamycin, clindamycin, finafloxacin, ofloxacin, ciprofloxacin, tobramycin, amikacin, mupirocin, penicillin, clarithromycin, metronidazole, cephalosporin, nafcillin, or vancomycin

2. A method for treating and curing bacterial infections of the skin, muscle, blood, or other bodily tissues in animals and humans afflicted bacterial infection caused by Methicillin-resistant Staphylococcus aureus comprising administering to the animal or human a therapeutically effective amount of artemisone in conjunction with an appropriate antibiotic in unit dosage form.

3. The method of claim 1, wherein the therapeutically effective amount of artemisone is between about 0.05-1000 mg per day.

4. The method of claim 1, wherein the duration of treatment of the animal or human lasts from 1 day to 60 days.

5. The method of claim 1, wherein the administration of artemisone in conjunction with appropriate antibiotic to the animal or human is via parenteral, oral or intraperitoneal administration.

6. The method of claim 1, wherein the unit dosage form of the oral administration is selected from the group consisting of hard or soft shell gelatin capsules, tablets, troches, sachets, lozenges, elixirs, suspensions, syrups, wafers, powders, granules, solutions and emulsions.

7. The method of claim 1, wherein the parenteral route of administration is selected from the group consisting of intravenous; intramuscular; interstitial; intra-arterial; subcutaneous; intraocular; intracranial; intraventricular; intrasynovial; transepithelial, including transdermal, pulmonary via inhalation, ophthalmic, sublingual and buccal; topical, including ophthalmic, dermal, ocular, rectal, and nasal inhalation via insufflation or nebulization.
